(19) United States
(12) Patent Application Publication
(10) Pub. No.: US 2010/0311158 A1
(43) Pub. Date: Dec. 9, 2010

(54) DEVICE AND SYSTEM FOR CULTURING CELLS AND METHOD OF CULTURING CELLS USING THE SAME

(76) Inventors: Sung Koo KANG, Gyunggi-do (KR); Hye Jin JIN, Jeollabuk-do (KR); Young-Ho CHO, Daejeon (KR); Young Soo OH, Gyunggi-do (KR); Sang Jin KIM, Gyunggi-do (KR); Bo Sung KU, Gyunggi-do (KR)

Correspondence Address: BLAKELY SOKOLOFF TAYLOR & ZAFMAN LLP, 1279 OAKMEAD PARKWAY, SUNNYVALE, CA, 94085-4040, US

(21) Appl. No.: 12/537,184

(22) Filed: Aug. 6, 2009

(30) Foreign Application Priority Data
Jun. 8, 2009 (KR) 10-2009-0050314

(51) Int. Cl. C12N 5/00; C12M 1/00

(52) U.S. Cl. 435/325; 435/289.1; 435/297.1; 435/287.1; 435/303.1

(57) ABSTRACT

The present invention provides a device for culturing cells. The device includes a well and a membrane. The well has an internal space and a membrane actuating control channel. The internal space has an inlet and an outlet. The membrane is provided between the membrane actuating control channel and the internal space such that the membrane actuating control channel is isolated from the internal space. The membrane is elastically varied in shape to form a three-dimensional cell culture space in the internal space.

### CROSS REFERENCE TO RELATED APPLICATION

This application claims the benefit of Korean Patent Application No. 10-2009-0050314, filed Jun. 8, 2009, entitled “DEVICE AND SYSTEM FOR CULTURING CELLS AND METHOD OF CULTURING CELLS USING THE SAME”, which is hereby incorporated by reference in its entirety into this application.

### BACKGROUND OF THE INVENTION

1. Field of the Invention

The present invention relates to a device and system for culturing cells and a method of culturing cells using the same.

2. Description of the Related Art

In the case of a conventional cell-based analysis, cells are adhered to a surface of a channel or well and thus form a film (2D). In this case, it is difficult to observe the actual behavior of cells.

In an effort to overcome the above problem, a method of forming or culturing 3D cell clusters in a fixed channel or well has been proposed. However, this method is disadvantageous in that it is not easy to extract cells from a cell culture device.

Therefore, in cases of a cell-based VTAS (micro analysis system), new drug screening or the like, a device and method of culturing cells which can culture a 3D cell structure to provide more precise information about the behavior of cells and which can facilitate the extraction of the cultured cells from the device are required.

### SUMMARY OF THE INVENTION

The present invention has been made in an effort to provide a device and system for culturing cells which can culture a 3D cell structure to provide precise information about the behavior of cells and which can facilitate the extraction of the cultured cells from the device, and a method of culturing cells using the same.

In a device for culturing cells according to an embodiment of the present invention, a well has an internal space and a membrane actuating control channel. The internal space has an inlet and an outlet. A membrane is provided between the membrane actuating control channel and the internal space such that the membrane actuating control channel is isolated from the internal space. The membrane is elastically varied in shape to form a cell culture space in the internal space.

The membrane actuating control channel may comprise a first membrane actuating control channel and a second membrane actuating control channel which are formed on opposite sides of the internal space. The membrane may comprise a first membrane provided between the first membrane actuating control channel and the internal space, and a second membrane provided between the second membrane actuating control channel and the internal space.

The membrane actuating control channel may have a shape which is open at a position corresponding to the inlet.

The well may have a polygonal shape, an arc shape or a shape of a combination of the polygonal shape and the arc shape.

The membrane actuating control channel may have a polygonal shape, a circular shape or a shape of a combination of the polygonal shape and the circular shape.

The membrane may be made of gas- or liquid-permeable material.

The device may further include a monitoring unit provided at one side of the well to monitor the cell culture space.

The device may further include a temperature control unit provided at one side of the well to control a temperature in the cell culture space.

The device may further include a cell analysis unit provided behind the outlet.

Furthermore, a surface treatment layer for preventing fixation of the cells or a proteinic surface treatment layer for promoting fixation of the cells may be applied to a surface of the well.

The device may further include an internal structure disposed in the internal space to adjust a rate of variation in shape of the membrane or to change a flow path of fluid in the internal space.

A system for culturing cells according to another embodiment of the present invention includes a plurality of the devices for culturing cells of claim 1. Wells or membrane actuating control channels of the plurality of devices for culturing cells are connected together to form a matrix shape.

A system for culturing cells according to a further embodiment of the present invention includes a plurality of devices for culturing cells of claim 1. Wells or membrane actuating control channels of the plurality of devices for culturing cells are connected in parallel to each other.

In a device of culturing cells according to yet another embodiment of the present invention, a device for culturing cells is provided. The device includes a well and a membrane. The well has an internal space which is provided with an inlet and an outlet, and a membrane actuating control channel. The membrane is disposed between the membrane actuating control channel and the internal space. A shape of the membrane is varied such that the membrane forms a cell culture space in the internal space. Cells are supplied into the cell culture space through the inlet. The cells are cultured. The cultured cells are discharged from the cell culture space through the outlet by varying a shape of the membrane.

In the culturing of the cells, three-dimensional cell clusters may be formed in the cell culture space. The three-dimensional cell clusters may be cultured.

The varying of the shape of the membrane may be conducted by increasing pressure in the membrane actuating control channel. The discharging of the cultured cells may be conducted by reducing pressure in the membrane actuating control channel.

Furthermore, a rate of variation in the shape of the membrane may be controlled by adjusting a thickness of the membrane or a cross-sectional area of a membrane actuating control channel.

The membrane may be made of gas- or liquid-permeable material, so that gas or liquid supplied into the membrane actuating control channel enters the cell culture space.

CO₂ gas, O₂ gas or a combination of CO₂ gas and O₂ gas may be supplied into the membrane actuating control channel to create a cell culture environment in the cell culture space.

### BRIEF DESCRIPTION OF THE DRAWINGS

The above and other objects, features and advantages of the present invention will be more clearly understood from the following detailed description taken in conjunction with the accompanying drawings, in which:

FIG. 1 is a plan view of a device for culturing cells, according to an embodiment of the present invention;

FIG. 2 is a sectional view of the cell culture device taken along the cutting plan line of FIG. 1;

FIG. 3 is a plan view of a cell culture device having a polygonal well, according to a modification of the present invention;

FIG. 4 is a plan view of a cell culture device in which internal structures are further provided in a well, according to another modification of the present invention;

FIG. 5 is a plan view of a cell culture device having a polygonal membrane actuating control channel, according to another modification of the present invention;

FIG. 6 is a sectional view of a cell culture device in which a membrane is set such that a rate of variation in shape changes depending on a region of the membrane according to the present invention;

FIG. 7 is a sectional view of a cell culture device having a first membrane actuating control channel and a second membrane actuating control channel, according to a modification of the present invention;

FIGS. 8 and 9 are views showing cell culture systems including a plurality of cell culture devices, according to the present invention; and

FIGS. 10 through 12 are views showing a method of culturing cells, according to an embodiment of the present invention.

### DESCRIPTION OF THE PREFERRED EMBODIMENTS

Hereinafter, a device and system for culturing cells and a method of culturing cells using the same according to an embodiment of the present invention will be described in detail with reference to the attached drawings. Reference will now be made to the drawings, which use the same reference numerals throughout the entirety thereof to designate the same or similar components. Note that in the following description of the embodiment of the present invention herein, words, such as “upper”, “lower”, etc., are used to merely distinguish components from each other and not to impose limits on the components.

Furthermore, the terms and words used in the specification and claims are not necessarily limited to typical or dictionary meanings, but must be understood to indicate concepts selected by the inventor as the best method of illustrating the present invention, and must be interpreted as having meanings and concepts adapted to the scope and spirit of the present invention for purposes of furthering understanding of the technology of the present invention.

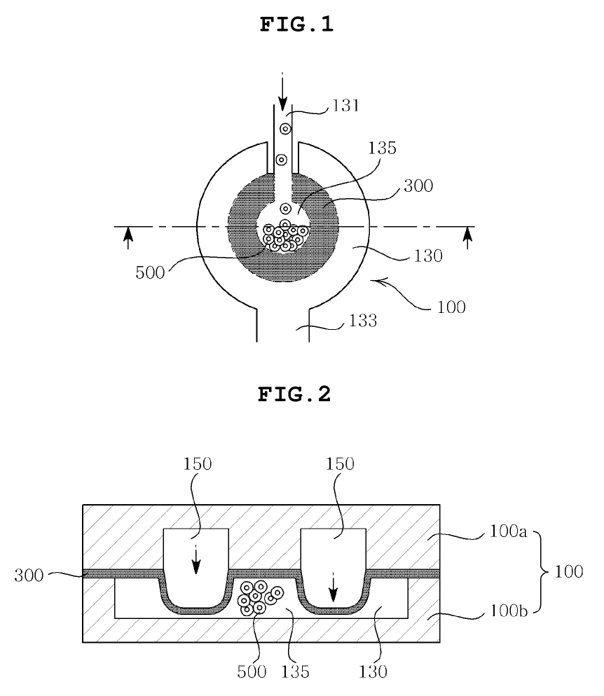

FIG. 1 is a plan view of a device for culturing cells, according to the embodiment of the present invention. FIG. 2 is a sectional view of the cell culture device taken along the cutting plan line of FIG. 1.

As shown in FIG. 1, the device for culturing cells according to the embodiment of the present invention includes a well 100 and a membrane 300. The well 100 defines an internal space 130 therein and has a membrane actuating control channel 150. The membrane 300 is disposed between the membrane actuating control channel 150 and the internal space 130.

The well 100 is an external structure which has the membrane actuating control channel 150 and the internal space 130 including an inlet 131 and an outlet 133. The well 100 may be formed into a single body or, alternatively, it may comprise an upper well 100a and a lower well 100b which are assembled together.

The well 100 is preferably made of biocompatible material. The material of the well 100 is not limited to a specific material, but it is preferable that it be made, for example, of PDMS, PMMA, biocompatible plastic, glasses, etc. Furthermore, a surface treatment layer for preventing fixation of cells 500 or a proteinic surface treatment layer for promoting fixation of cells 500 is applied to the surface of the well 100.

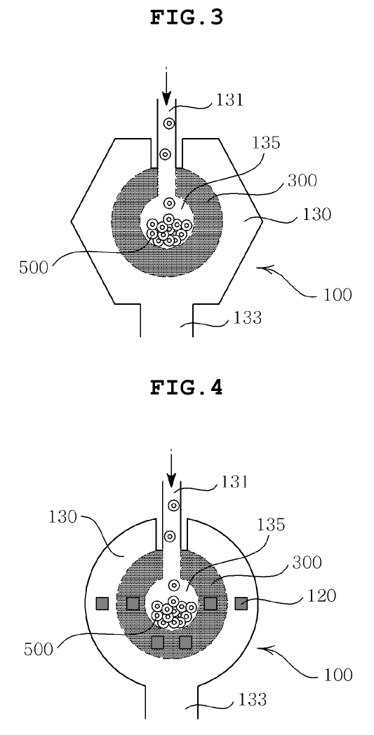

The shape of the well 100 is not limited. As shown in FIG. 1, the well 100 may have a circular shape or, alternatively, it may have a polygonal shape, as shown in FIG. 3. As a further alternative, although it is not shown in the drawings, the well 100 may have an arc shape, or it may have a shape of a combination of these shapes.

As shown in FIG. 4, internal structures 120 may be provided in the internal space 130 of the well 100 to adjust a rate of variation in shape of the membrane 300 or change a flow path of fluid in the internal space 130. The heights of the internal structures 120 are determined in consideration of a degree of variation of the membrane 300. The locations of the internal structures 120 in the internal space 130 are determined in consideration of a moving path along which cells 500 and fluid flow into the internal space 130.

The internal space 130 defined in the well 100 is an area in which a cell culture space 135 which will be explained later is formed by the membrane 300. The internal space 130 is connected with the inlet 131 and the outlet 133 which communicate with outside of the well 100. The inlet 131 and the outlet 133 provide a path along which the cells 500 and cell culture fluid are supplied into and discharged from the internal space 130. The inlet 131 and the outlet 133 may be formed by holes which are formed in the well 100 or, alternatively, they may comprise hollow pipes which extend outwards from the well 100.

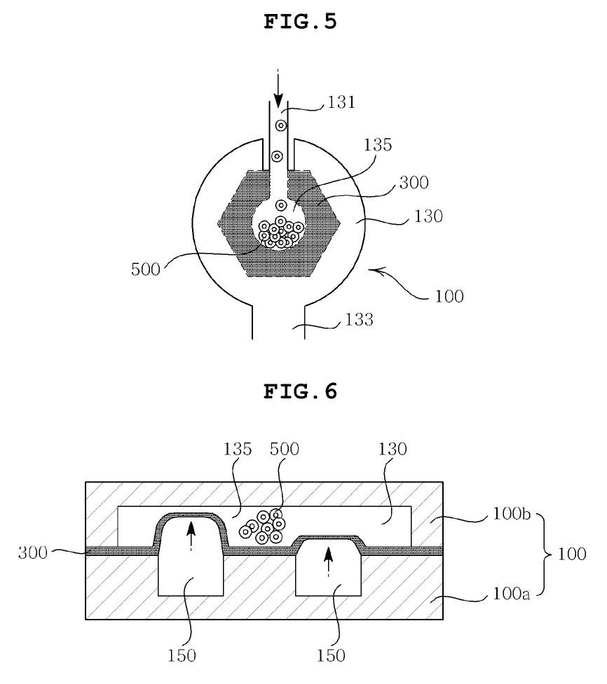

The membrane actuating control channel 150 functions to control a rate of variation in the shape of the membrane 300. In addition, the membrane actuating control channel 150 may serve as a path along which fluid is drawn into the cell culture space 135 to make cell culture circumstances in the cell culture space 135. As shown in FIG. 1, the membrane actuating control channel 150 may have a circular shape or, alternatively, it may have a polygonal shape, as shown in FIG. 5. As a further alternative, although it is not shown in the drawings, the membrane actuating control channel 150 may assume an arc shape, or it may assume a shape that is a combination of these shapes. In other words, the shape of the membrane actuating control channel 150 is not limited to a specific shape. Furthermore, the membrane actuating control channel 150 may have a shape in which it blocks the outlet 133, rather than having a shape surrounding the cell culture space 135. Merely, it is preferable that the membrane actuating control channel 150 have a ‘C’ shape which is open at a position corresponding to the inlet passes such that the open state of the inlet 131 is ensured. The shape of the membrane actuating control channel 150 becomes a factor which defines the shape of the cell culture space 135 which will be explained later.

The membrane 300 elastically varies in shape and defines the cell culture space 135 in the internal space 130 of the well 100. The membrane 300 is disposed between the membrane actuating control channel 150 and the internal space 130 such that the membrane actuating control channel 150 is isolated from the internal space 130.

The membrane 300 may comprise an elastomeric membrane (a deformable membrane) which is made, for example, of a PDMS, natural rubber, synthetic polymer latex, soft or hard rubber or plastic. Preferably, the membrane 300 is made of gas or liquid-permeable material to provide superior cell culture circumstances in the cell culture space 135.

The method of varying the shape of the membrane 300 is not limited to a specific method. For example, a hydraulic method, a pneumatic method, a piezoelectric actuating method, a thermal actuating method, an electrostatic actuating method or an electromagnetic actuating method can be used. In the embodiment, a pneumatic method is used as the method of varying the shape of the membrane 300. In detail, when gas is supplied into the membrane actuating control channel 150, the pressure in the membrane actuating control channel 150 is increased. Thus, the membrane 300 elastically expands. Because the membrane actuating control channel 150 has a closed curve shape other than the inlet 131, the three-dimensional cell culture space 135 having a predetermined volume is formed in the membrane 300 that elastically expands. In this state, if the pressure in the membrane actuating control channel 150 is maintained, the membrane 300 maintains the expanded state. Thereby, the cells 500 contained in the cell culture space 135 can be prevented from leaving the cell culture space 135 through the outlet 133. In this state, if the pressure in the membrane actuating control channel 150 is reduced, the membrane 300 is contracted by its inherent elasticity, so that the internal space 130 communicates with the outlet 133. A rate of variation in shape of the membrane 300 can be controlled by adjusting the thickness of the membrane 300 and the cross-sectional area and width of the membrane actuating control channel 150. As shown in FIG. 6, the membrane 300 is set such that a rate of variation in shape varies depending on a region of the membrane 300 (for example, rates of variation of the left portion and the right portion in FIG. 6 differ from each other to have an asymmetrical structure).

In the above embodiment, although the single membrane actuating control channel 150 has been illustrated as being provided in the upper part of the well 100, two or more membrane actuating control channels 150 may be formed in the well 100, and the membrane actuating control channel 150 may be formed in the lower part of the well 100.

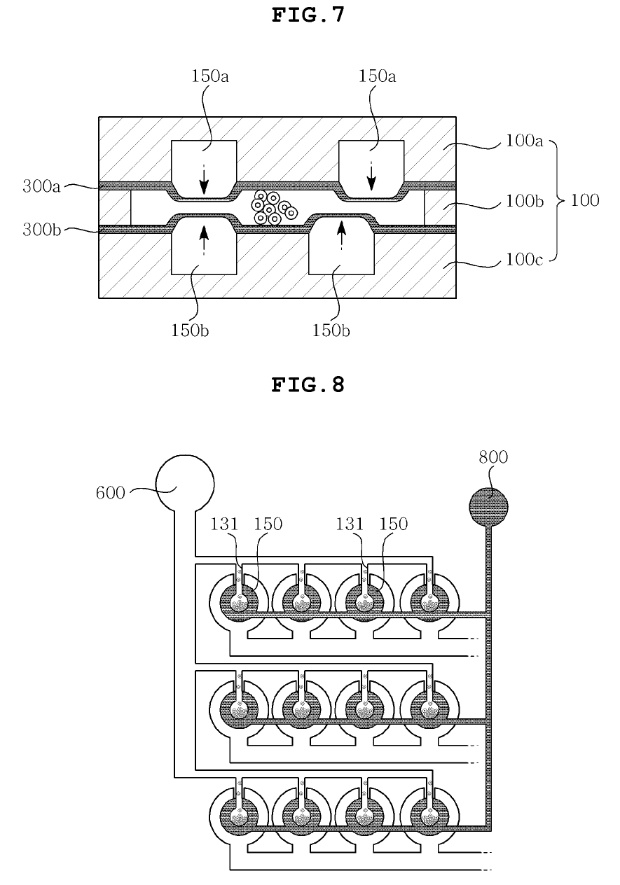

For example, as shown in FIG. 7, a well 100 may include a first membrane actuating control channel 150a and a second membrane actuating control channel 150b which are formed on opposite sides of an internal space 130. In this case, membranes 300a and 300b which respectively correspond to the first and second membrane actuating control channels 150a and 150b are provided in the well 100. In other words, the first membrane 300a is provided between the first membrane actuating control channel 150a and the internal space 130. The second membrane 300b is provided between the second membrane actuating control channel 150b and the internal space 130. Furthermore, the well 100 may be divided into three parts including an upper well 100a, a middle well 100b and a lower well 100c.

Meanwhile, the cell culture device according to the embodiment of the present invention may further include a monitoring unit which is disposed at one side of the well 100 to monitor the cell culture space 135, and a temperature control unit which is provided at one side of the well 100 to control the temperature in the cell culture space 135. The cell culture device may further include a homogenization module or a fluid distribution module which is provided in front of the inlet 131, and a cell analysis unit which is provided in back of the outlet 133 to analyze genes, protein, etc. of the cells 500. These constructions use well-known techniques, so their illustration and description will be omitted.

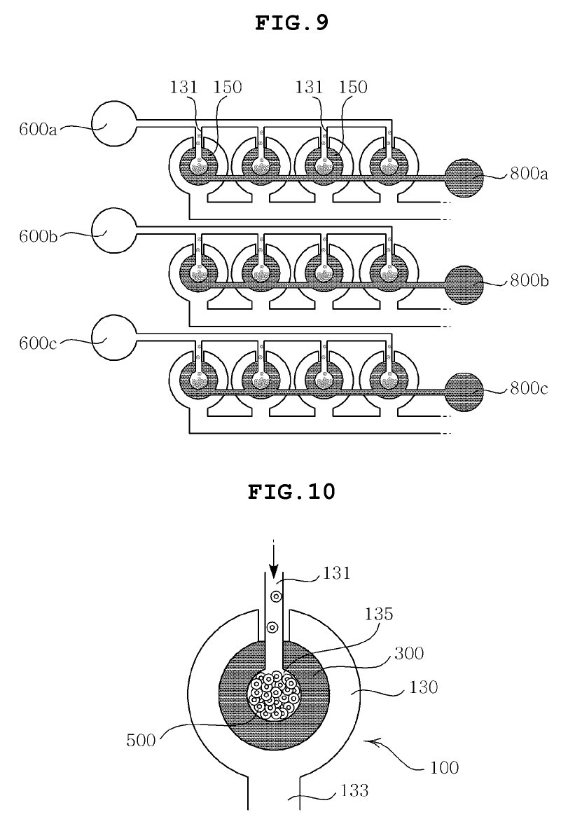

FIGS. 8 and 9 are views showing systems for culturing cells including a plurality of cell culture devices, according to the present invention.

As mentioned above, a single cell culture device may be used as a single unit, but a plurality of cell culture devices may be connected to each other to construct a cell culture system.

FIG. 8 is a view illustrating a system culturing cells. In this system for culturing cells, several lines each of which includes cell culture devices connected in parallel to each other are arranged. All inlets of the cell culture devices are connected to a single cell supply unit 600. That is, the cell culture devices are connected to each other in a matrix shape. In this case, as shown in the drawing, all membrane actuating control channels 150 formed in the cell culture devices may also be connected to a single control unit 800.

FIG. 9 is a view illustrating another system for culturing cells. In this system for culturing cells, several lines each of which include cell culture devices connected in parallel to each other are arranged. Inlets of cell culture devices of each line are connected to a corresponding cell supply unit 600a, 600b, 600c which is provided on each line. That is, FIG. 9 shows the cell culture system in which each line has the cell culture devices which are connected in parallel to each other. In this case, as shown in the drawing, membrane actuating control channels 150 of the cell culture devices which are provided on each line may be connected in parallel to a corresponding control unit 800a, 800b, 800c which is provided on each line.

Here, the connection between the inlets 131 and the cell supply units 600a, 600b and 600c is independent from the connection between the membrane actuating control channels 150 and the control units 800a, 800b and 800c. The inlets 131 may be connected to each other in a matrix shape and the membrane actuating control channels 150 may be connected in parallel to each other, and vice-versa.

In the cell culture device described above, because the cell culture space 135 is defined by the membrane 300 which elastically varies in shape, three-dimensional cells 500 which are cultured in the cell culture space 135 can be easily extracted from the cell culture space 135 by a simple manipulation which adjusts a rate of variation in shape of the membrane 300.

Furthermore, since the membrane 300 which forms the cell culture space 135 is made of gas or liquid-permeable material, the present invention can easily create superior culture circumstances in the cell culture space 135 and culture cells 500 over a long period of time.

Moreover, in the present invention, the volume of the cell culture space 135 can be easily controlled by adjusting a rate of variation in shape of the membrane 300. Thus, it is easy to control the size of a tissue of the cultured cells 500.

Hereinafter, a method of culturing cells according to an embodiment of the present invention will be described in detail with reference to FIGS. 10 through 12.

First, is prepared the cell culture device which includes the well 100 and the membrane 300, the well 100 having the internal space 130 including the inlet 131 and the outlet 133 and the membrane actuating control channel 150, the membrane 300 being disposed between the membrane actuating control channel 150 and the internal space 130. The shape of the membrane 300 is varied such that the membrane 300 forms the cell culture space 135 in the internal space 130.

The construction of the cell culture device is as described above. In the embodiment, the variation in shape of the membrane 300 is conducted by increasing pneumatic pressure in the membrane actuating control channel 150. Furthermore, a rate of variation in shape of the membrane 300 can be controlled by adjusting the thickness of the membrane 300 or the cross-sectional area of the membrane actuating control channel 150.

Thereafter, as shown in FIG. 10, cells 500 are supplied into the cell culture space 135 through the inlet 131. At this time, because the outlet 133 is in the state of being closed by the membrane 300 such that the cells 500 are prevented from passing through the outlet 133, the cells 500 that are supplied into the cell culture space 135 through the inlet 131 can stably remain in the cell culture space 135 without leaking through the outlet 133.

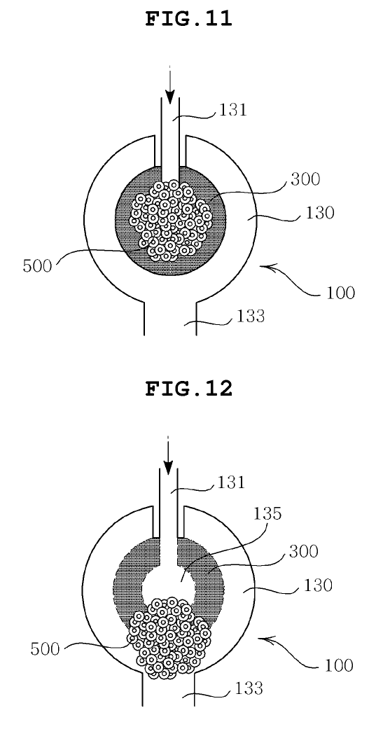

Subsequently, as shown in FIG. 11, the cells 500 are cultured. In the state in which culture circumstances are created in the cell culture space 135, the supplied cells 500 form three-dimensional cell clusters 500.

Culture fluid required for culturing the cells 500 is supplied through the inlet 131. Surplus culture fluid and secreted cells 500 are discharged to the outlet 133 through a gap between the membrane 300 and the well 100. Furthermore, the membrane 300 is made of gas or liquid-permeable material, so that gas or liquid supplied into the membrane actuating control channel 150 enters the cell culture space 135, thus providing superior culture circumstances. For example, CO₂ gas, O₂ gas or a combination of these may be supplied into the membrane actuating control channel 150 to control the pH of the cell culture space 135 or the partial pressure of the gas distribution.

Thereafter, as shown in FIG. 12, the cells 500 are extracted from the cell culture space 135 through the outlet 133 by varying the shape of the membrane 300. In detail, when the pressure of fluid supplied into the membrane actuating control channel 150 is reduced, the membrane 300 is elastically contracted. Thereby, the outlet 133 is opened such that it communicates with the cell culture space 135. Thus, the cultured cells 500 are discharged through the outlet 133. Furthermore, the discharge of the cells 500 can be more easily conducted by creating a difference in pressure between the inlet 131 and the outlet 133.

In the method of culturing cells according to the present invention, the cell culture space 135 which has been isolated from the outlet 133 by elastically varying the shape of the membrane 300 can be connected to the outlet 133 by a simple manipulation in which the membrane 300 which has been deformed is returned to its original state. Hence, the operation of extracting the cells 500 from the cell culture space 135 can be markedly simplified.

Furthermore, because the environment of the cell culture is controlled through the membrane 300 which forms the cell culture space 135, a superior culturing environment can be easily created, and the cells 500 can be cultured over a long period of time.

In addition, since the volume of the cell culture space 135 can be controlled by adjusting a rate of variation in shape of the membrane 300, the size of the cultured cell tissue can be easily controlled.

As described above, in the present invention, because a cell culture space is formed by a membrane which is elastically deformable, cells which are cultured in the cell culture space can be easily extracted from the cell culture space by simple manipulation whereby a rate of variation in shape of the membrane is adjusted.

Furthermore, the membrane which forms the cell culture space is made of gas or liquid-permeable material. Thus, the present invention can easily create a superior culturing environment in the cell culture space and culture cells over a long period of time.

Moreover, since the volume of the cell culture space can be controlled by adjusting a rate of variation in shape of the membrane, the size of the cultured cell tissue can be easily controlled.

Although the preferred embodiment of the present invention has been disclosed for illustrative purposes, those skilled in the art will appreciate that various modifications, additions and substitutions are possible, without departing from the scope and spirit of the invention as disclosed in the accompanying claims. Therefore, these various modifications, additions and substitutions must be regarded as falling within the bounds of the claims of the present invention.

What is claimed is:

1. A device for culturing cells, comprising:
a well having an internal space and a membrane actuating control channel, the internal space having an inlet and an outlet; and
a membrane provided between the membrane actuating control channel and the internal space such that the membrane actuating control channel is isolated from the internal space, the membrane being elastically varied in shape to form a cell culture space in the internal space.

2. The device as set forth in claim 1, wherein
the membrane actuating control channel comprises a first membrane actuating control channel and a second membrane actuating control channel which are formed on opposite sides of the internal space, and
the membrane comprises a first membrane provided between the first membrane actuating control channel and the internal space, and a second membrane provided between the second membrane actuating control channel and the internal space.

3. The device as set forth in claim 1, wherein the membrane actuating control channel has a shape which is open at a position corresponding to the inlet.

4. The device as set forth in claim 1, wherein the well has a polygonal shape, an arc shape or a shape of a combination of the polygonal shape and the arc shape.

5. The device as set forth in claim 1, wherein the membrane actuating control channel has a polygonal shape, a circular shape or a shape of a combination of the polygonal shape and the circular shape.

6. The device as set forth in claim 1, wherein the membrane is made of gas- or liquid-permeable material.

7. The device as set forth in claim 1, further comprising:
a monitoring unit provided at one side of the well to monitor the cell culture space.

8. The device as set forth in claim 1, further comprising:
a temperature control unit provided at one side of the well to control a temperature in the cell culture space.

9. The device as set forth in claim 1, further comprising:
a cell analysis unit provided behind the outlet.

10. The device as set forth in claim 1, wherein a surface treatment layer for preventing fixation of the cells or a proteinic surface treatment layer for promoting fixation of the cells is applied to a surface of the well.

11. The device as set forth in claim 1, further comprising:
an internal structure disposed in the internal space to adjust a rate of variation in shape of the membrane or to change a flow path of fluid in the internal space.

12. A system for culturing cells, comprising: a plurality of the devices for culturing cells of claim 1,
wherein wells or membrane actuating control channels of the plurality of devices for culturing cells are connected together to form a matrix shape.

13. A system for culturing cells, comprising: a plurality of devices for culturing cells of claim 1,
wherein wells or membrane actuating control channels of the plurality of devices for culturing cells are connected in parallel to each other.

14. A method of culturing cells, comprising:
providing a device for culturing cells, comprising a well and a membrane, the well having an internal space provided with an inlet and an outlet, and a membrane actuating control channel, the membrane being disposed between the membrane actuating control channel and the internal space;
varying a shape of the membrane such that the membrane forms a cell culture space in the internal space;
supplying cells into the cell culture space through the inlet;
culturing the cells; and
discharging the cultured cells from the cell culture space through the outlet by varying a shape of the membrane.

15. The method as set forth in claim 14, wherein the culturing of the cells comprises:
forming three-dimensional cell clusters in the cell culture space; and
culturing the three-dimensional cell clusters.

16. The method as set forth in claim 14, wherein the varying of the shape of the membrane is conducted by increasing pressure in the membrane actuating control channel, and the discharging the cultured cells is conducted by reducing pressure in the membrane actuating control channel.

17. The method as set forth in claim 14, wherein a rate of variation in the shape of the membrane is controlled by adjusting a thickness of the membrane or a cross-sectional area of a membrane actuating control channel.

18. The method as set forth in claim 14, wherein the membrane is made of gas- or liquid-permeable material, so that gas or liquid supplied into the membrane actuating control channel enters the cell culture space.

19. The method as set forth in claim 14, wherein CO₂ gas, O₂ gas or a combination of CO₂ gas and O₂ gas is supplied into the membrane actuating control channel to create a cell culture environment in the cell culture space.
